(19) United States
(12) Patent Application Publication
(10) Pub. No.: US 2011/0054193 A1
(43) Pub. Date: Mar. 3, 2011

(54) STATIN PRODUCTION

(76) Inventors: Marco Alexander Van Den Berg, Poeldijk (NL); Marcus Hans, Den Haag (NL)

(21) Appl. No.: 12/989,540

(22) PCT Filed: Apr. 28, 2009
(86) PCT No.: PCT/EP09/55102

§ 371 Date: Oct. 25, 2010

(30) Foreign Application Priority Data
Apr. 29, 2008 (EP) 08155372.9

(51) Int. Cl. C07D 309/30; C12N 9/02; C07H 21/04; C12P 17/06; C12P 7/64; C12N 1/15; C12N 1/19; C07C 69/34

(52) U.S. Cl. 549/292; 435/189; 536/23.2; 435/125; 435/134; 435/254.5; 435/254.3; 435/254.8; 435/254.11; 435/254.21; 560/194

(57) ABSTRACT

The present invention relates to a polypeptide with HMG-CoA reductase activity, to its polynucleotide congener and to a method for the production of a statin comprising over expression of said polypeptide.

### FIELD OF THE INVENTION

The present invention relates to a method for fermentation of statins.

### BACKGROUND OF THE INVENTION

Cholesterol and other lipids are transported in body fluids by low-density lipoproteins (LDL) and high-density lipoproteins (HDL). Substances that effectuate mechanisms for lowering LDL-cholesterol may serve as effective antihypercholesterolemic agents because LDL levels are positively correlated with the risk of coronary artery disease. Cholesterol lowering agents of the statin class are medically very important drugs as they lower the cholesterol concentration in the blood by inhibiting HMG-CoA reductase. The latter enzyme catalyses the rate limiting step in cholesterol biosynthesis, i.e. the conversion of (3S)-hydroxy-3-methylglutarylcoenzyme A (HMG-CoA) to mevalonate. As can be seen from the scheme below, there are several types of statins on the market, amongst which atorvastatin, compactin (1), lovastatin (3), simvastatin (4) and pravastatin (6). Whilst atorvastatin is made via chemical synthesis, the other statins mentioned above are produced either via direct fermentation or via precursor fermentation. These (precursor) fermentations are carried out by fungi of the genera Penicillium, Aspergillus and Monascus.

There is a common problem while fermenting these compounds in fungi as the final products are besides cholesterol lowering agents also active antifungals (see for example Qiao, J., Kontoyiannis, D. P., Wan, Z., Li, R. and Liu, W., Med. Mycol. 2007, 45:589-593) and thereby limit the productivity in fungal hosts. A possible solution to this problem could be the transfer of the metabolic pathway to bacterial species, which might be less sensitive to statins. However, this solution is not easy as two fungal polyketide synthases are part of the statin metabolic pathways and these are quite different from bacterial polyketide synthases. In fact, examples of cross kingdom expression are limited to single and simple fungal polyketide synthases as in the synthesis of 6-methyl salicylic acid (6-MSA) from Penicillium patulum in Streptomyces (Bedford, D. J., Schweizer, E., Hopwood, D. A. and Khosla, C., J. Bacteriol. 1995, 177:4544-4548) and result in very low titers of 60 mg/liter, while fungal statin fermentations lead to multi grams per liter. Even heterologous production of bacterial polyketides in a bacterium is tough and there are only limited examples where this worked properly (see for example (Lau et al., J. Biotechnology 2004, 110:95-103). Hence, there is a need for improvement of the productivity of fungal fermentations due the anti-fungal properties of statins.

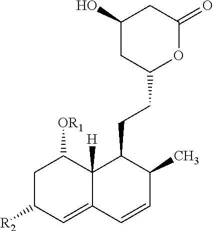

| | R₁ | R₂ |
|---|---|---|
| 1 (compactin) | 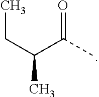 | H |
| 2 (ML-236A) | H | H |
| 3 (lovastatin) | 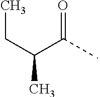 | CH₃ |
| 4 (simvastatin) | 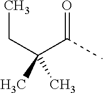 | CH₃ |
| 5 (monacolin J) | H | CH₃ |
| 6 (pravastatin) | 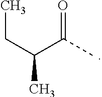 | OH |
| 7 | H | OH |

### DESCRIPTION OF THE INVENTION

The object of the present invention is to provide a method to solve some of the problems encountered in prior art processes. Thus, provided is a method in which the sensitivity of the production host to statins is decreased by genetic engineering. More specifically a process for increasing the compactin, pravastatin, lovastatin and/or simvastatin productivity is provided, characterized in that the fermentation process is carried out with hosts that are engineered to have increased resistance to compactin, pravastatin, lovastatin and/or simvastatin. Preferably, a process is provided which makes use of microorganisms in which genes encoding proteins mediating statin resistance are over expressed.

In the context of the present invention compactin, pravastatin, lovastatin and/or simvastatin (generally referred to as ‘statin’ or ‘statins’) ‘biosynthetic genes’ include all genes encoding enzymes directly involved in the synthesis of statin molecules, all genes encoding enzymes in secretion of statin molecules and all genes encoding proteins involved in the transcriptional regulation of the genes of the first two categories. Also, included are all genes of the microbial host capable of producing statins which by over expression or inactivation cause a significant change in the production capacity (i.e. resulting in at least 20% more statin produced or in at least 20% less statin produced, respectively). Specific genes are, but not limited to: the compactin biosynthetic gene cluster of Penicillium citrinum (i.e. mlcA, mlcB, mlcC, mlcD, mlcE, mlcF, mlcH, mlcG, mlcR; see Entrez database accession number AB072893; Abe Y, Suzuki T, Ono C, Iwamoto K, Hosobuchi M and Yoshikawa H, Mol Genet Genomics 2002, 267:636-646), the lovastatin biosynthetic gene cluster of Aspergillus terreus (i.e. ORF1, ORF2, lovA, ORF5, lovC, lovD, ORF8, lovE, ORF10, lovF, ORF12, ORF13, ORF14, ORF15, ORF16, cytochrome P450 monooxygenase, ORF18; see Entrez database accession numbers AF141924 and AF141925; Kennedy J, Auclair K, Kendrew S G, Park C, Vederas J C and Hutchinson C R, Science 1999, 284:1368-1372), the monacolin K biosynthetic gene cluster of Monascus pilosus (i.e. mkA, mkB, mkC, mkD, mkE, mkF, mkG, mkH and mkI; see Entrez database accession number DQ176595, http://vvww.ncbi.nlm.nih.gov/entrez/viewer.fcgi?db=nuccore&id=74275560), and substantial homologues thereof originating from other species.

In the context of this invention the terms ‘over expressed’ and/or ‘over expression’ are used to describe the various methods by which a gene or a protein can be modified in order to produce more active enzyme. This includes: introduction of additional gene copies encoding host or heterologous proteins; over expression of host proteins from a strong promoter; modifying the transcriptional regulation of the genes encoding enzymes mediating statin resistance; mutation of critical amino acids leading to proteins with improved kinetic properties; mutations causing a increased half-life of the enzyme; modifying the mRNA molecule in such away that the mRNA half-life is increased; modifying the intracellular localization of the protein towards an organelle in which no statins are present to inhibit its activity; introduction of one or more copies of heterologous genes encoding enzymes mediating statin resistance; inactivation of genes and/or proteins mediating sensitivity towards statins. Preferably over expression is obtained by introducing additional gene copies or driving gene transcription from a strong promoter. Most preferably increased resistance towards statins is obtained by over expression of the proteins of the current invention.

In the context of this invention the terms ‘inactivated’ and/or ‘inactivation’ are used to describe the various methods by which a gene or a protein can be modified in order to produce less active enzyme. This includes: inactivation by base pair mutation resulting in a(n early) stop or frame shift; mutation of critical amino acids; mutations causing a decreased half-life of the enzyme; modifying the mRNA molecule in such away that the mRNA half-life is decreased; insertion of a second sequence (i.e. a selection marker gene) disturbing the open reading frame; a partial or complete removal of the gene; removal/mutation of the promoter of the gene; using anti-sense DNA or comparable RNA inhibition methods to lower the effective amount of mRNA in the cell.

In the context of the present invention the term ‘mediating’ is used to describe the various functions by which a gene or a protein can cause resistance or sensitivity towards statins. This includes: active or passive secretion of statins; modification of the membrane structure to influence the diffusion of statins; increased protein numbers of inhibited enzymes to allow for proper catalytic function in the cell; intracellular transport of statins towards specific organelles.

In the context of the present invention, the term “conservative substitution” is intended to mean that a substitution in which the amino acid residue is replaced with an amino acid residue having a similar side chain. These families are known in the art and include amino acids with basic side chains (e.g. lysine, arginine and histidine), acidic side chains (e.g. aspartic acid, glutamic acid), uncharged polar side chains (e.g., glycine, asparagines, glutamine, serine, threonine, tyrosine, cysteine), non-polar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine tryptophan, histidine).

The term “isolated polynucleotide or nucleic acid sequence” as used herein refers to a polynucleotide or nucleic acid sequence which is essentially free of other nucleic acid sequences, e.g., at least 20% pure, preferably at least 40% pure, more preferably at least 60% pure, even more preferably at least 80% pure, most preferably at least 90% pure as determined by agarose electrophoresis. For example, an isolated nucleic acid sequence can be obtained by standard cloning procedures used in genetic engineering to relocate the nucleic acid sequence from its natural location to a different site where it will be reproduced.

In a first aspect, provided is a polypeptide selected from the group consisting of a polypeptide having an amino acid sequence according to SEQ ID NO 4 and a polypeptide having an amino acid that is substantially homologous to the sequence of SEQ ID NO 12, the polypeptide displaying 3-hydroxy-3-methyl-glutaryl-CoenzymeA reductase (HMGR) activity.

In a first embodiment, said polypeptide converts HMG into mevalonate. The enzyme belongs to the class of EC1.1.1.88 or EC1.1.1.34. A polypeptide with an amino acid sequence that is substantially homologous to SEQ ID NO 4 is defined as a polypeptide with an amino acid sequence with a degree of identity to the specified amino acid sequence of at least 80%, preferably at least 85%, more preferably at least 90%, still more preferably at least 95%, still more preferably at least 97%, still more preferably at least 98%, most preferably at least 99%. A polypeptide with an amino acid sequence that is substantially homologous to SEQ ID NO 12 is defined as a polypeptide with an amino acid sequence with a degree of identity to the specified amino acid sequence of at least 60%, preferably at least 70%, more preferably at least 80%, still more preferably at least 85%, still more preferably at least 90%, still more preferably at least 95%, still more preferably at least 97%, still more preferably least 98%, most preferably at least 99%. A substantially homologous polypeptide encompasses polymorphisms that may exist in cells from different populations or within a population due to natural allelic or intra-strain variation. A substantially homologous polypeptide may further be derived from a species other than the species where the specified amino acid and/or DNA sequence originates from, or may be encoded by an artificially designed and synthesized DNA sequence. DNA sequences related to the specified DNA sequences and obtained by degeneration of the genetic code are also part of the invention. Homologues also encompass biologically active fragments of the full-length sequence, still displaying HMGR activity. Also, larger proteins of which a part is substantially homologous to either SEQ ID NO 4 or SEQ ID NO 12 and display HMR activity are considered part of this invention.

The degree of identity between two amino acid sequences refers to the percentage of amino acids that are identical between the two sequences. The degree of identity is determined using the BLAST algorithm, which is described in Latched et al. (J. Mol. Biol. 1990, 215:403-410). BLAST analysis software is available through the National Center for Biotechnology Information (http://www.ncbi.nlm.nih.gov/). The BLAST algorithm parameters W, T, and X determine the sensitivity and speed of the alignment. The BLAST program uses as defaults a word length (W) of 11, the BLOSUM62 scoring matrix (see Henikoff and Henikoff, 1989, Proc Natl. Acad. Sci. USA 89:10915) alignments (B) of 50, expectation (E) of 10, M=5 and N=−4.

Substantially homologous polypeptides may contain only conservative substitutions of one or more amino acids of the specified amino acid sequences or substitutions, insertions or deletions of non-essential amino acids. Accordingly, a non-essential amino acid is a residue that can be altered in one of these sequences without substantially altering the biological function. For example, guidance concerning how to make phenotypically silent amino acid substitutions is provided in Bowie et al. (Science 1990, 247:1306-1310) indicating that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. These studies have revealed that proteins are surprisingly tolerant to amino acid substitutions and reveal which changes are likely to be permissive at a certain position of the protein. For example, most buried amino acid residues require non-polar side chains, whereas few features of surface side chains are generally conserved. Other such phenotypically silent substitutions are described in Bowie et al, and the references cited therein.

In a second embodiment, variants of the amino acid sequences of the present inventions leading to an “improved catalytic function” (i.e. HMGR activity) may be obtained by modifying the corresponding genes of the present invention. In the context of this invention such an ‘improved catalytic function’ is not limited to features like Kcat, Km, temperature optimum, half-life, turnover number, but may very well be a variant which is more resistant towards one or more of the statins, with the same HMGR activity compared to the parent protein. Among such modifications are:
1. Error prone PCR to introduce random mutations, followed by a screening of obtained variants and isolating of variants with improved kinetic properties
2. Family shuffling of related variants of the genes encoding HMGR enzyme(s), followed by a screening of obtained variants and isolating of variants with improved kinetic properties
3. Mutation of the serine residue, normally the target of phosphorylation by SNF1-related protein kinase 1, SnRK1 (see for details Hey et al., Plant Biotechnol. J. 2006, 4:219-229)
4. Targeted modification of binding sites for sterol-accelerated degradation, mediated by specific factors like insig-1 (see for details Sever et al., Mol. Cell. 2003, 11:25-33 and Xu and Simoni, Arch. Biochem. Biophys. 2003, 414:232-243)

In the context of this invention ‘improved genes’ are variants of the genes of the present invention leading to an increased level of mRNA and/or protein, resulting in more enzyme activity (i.e. HMGR activity). These may be obtained by modifying the polynucleotide sequences of said genes. Among such modifications are:
1. Improving the codon usage in such a way that the codons are (optimally) adapted to the parent microbial host.
2. Improving the codon pair usage in such a way that the codons are (optimally) adapted to the parent microbial host
3. Addition of stabilizing sequences to the genomic information encoding the HMGR enzyme(s) resulting in mRNA molecules with an increased half life

Preferred methods to isolate variants with improved catalytic properties or increased levels of mRNA or protein are described in WO03010183 and WO0301311. Preferred methods to optimize the codon usage in parent microbial strains are described in PCT/EP2007/05594. Preferred methods to add stabilizing elements to the genes encoding the HMGR enzyme(s) are described in WO2005059149.

In a third embodiment, there is provided a polynucleotide or nucleic acid sequence comprising a DNA sequence encoding the polypeptides mentioned above. This may be an isolated polynucleotide of genomic, cDNA, RNA, semi-synthetic, synthetic origin, or any combinations thereof. In particular, a specific DNA sequence is provided encoding the polypeptide of SEQ ID NO 4, i.e. SEQ ID NO 1, 2 or 3 and a specific DNA sequence is provided encoding the polypeptide of SEQ ID NO 12, i.e. SEQ ID NO 9, 10 or 11. The scope of the invention is not limited to these sequences, but includes substantially homologous polynucleotides encoding enzymes with HMGR activity. A polynucleotide with a nucleotide sequence that is substantially homologous to SEQ ID NO 1 is defined as a polynucleotide with a nucleotide sequence with a degree of identity to the specified nucleotide sequence of at least 80%, preferably at least 85%, more preferably at least 90%, still more preferably at least 95%, still more preferably at least 97%, still more preferably at least 98%, most preferably at least 99%. A polynucleotide with a nucleotide sequence that is substantially homologous to SEQ ID NO 2 is defined as a polynucleotide with a nucleotide sequence with a degree of identity to the specified nucleotide sequence of at least 80%, more preferably at least 85%, still more preferably at least 90%, still more preferably at least 95%, still more preferably at least 97%, still more preferably at least 98%, most preferably at least 99%. A polynucleotide with a nucleotide sequence that is substantially homologous to SEQ ID NO 3 is defined as a polynucleotide with a nucleotide sequence with a degree of identity to the specified nucleotide sequence of at least 85%, preferably at least 90%, still more preferably at least 95%, still more preferably at least 97%, still more preferably at least 98%, most preferably at least 99%. A polynucleotide with a nucleotide sequence that is substantially homologous to SEQ ID NO 9 is defined as a polynucleotide with a nucleotide sequence with a degree of identity to the specified nucleotide sequence of at least 60%, preferably at least 70%, more preferably at least 80%, still more preferably at least 90%, still more preferably at least 95%, still more preferably at least 97%, still more preferably at least 98%, most preferably at least 99%. A polynucleotide with a nucleotide sequence that is substantially homologous to SEQ ID NO 10 is defined as a polynucleotide with a nucleotide sequence with a degree of identity to the specified nucleotide sequence of at least 60%, preferably at least 70%, more preferably at least 80%, still more preferably at least 90%, still more preferably at least 95%, still more preferably at least 97%, still more preferably at least 98%, most preferably at least 99%. A polynucleotide with a nucleotide sequence that is substantially homologous to SEQ ID NO 11 is defined as a polynucleotide with a nucleotide sequence with a degree of identity to the specified nucleotide sequence of at least 60%, preferably at least 70%, more preferably at least 80%, still more preferably at least 90%, still more preferably at least 95%, still more preferably at least 97%, still more preferably at least 98%, most preferably at least 99%.

Unless otherwise indicated, all nucleotide sequences determined by sequencing a DNA molecule herein are determined using an automated DNA sequencer and all amino acid sequences of polypeptides encoded by DNA molecules determined herein were predicted by translation of a DNA sequence determined as above. Therefore, for any DNA sequence determined by this automated approach, any nucleotide sequence determined may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion. The person skilled in the art is capable of identifying such erroneously identified bases and knows how to correct for such errors.

The polypeptides and the encoding nucleic acid sequences of the first aspect of the invention may be obtained from any cell, preferably from cells which are highly resistant towards statins. Preferred species include, but are not limited to, strains of Aspergillus, Penicillium, Monascus, Streptomyces and Pseudomonas. In a preferred embodiment, the nucleic acid sequence encoding a polypeptide of the present invention is obtained from a strain of Penicillium chrysogenum.

DNA sequences of the invention may be identified by hybridization. Nucleic acid molecules corresponding to variants (e.g. natural allelic variants) and homologues of the DNA of the invention can be isolated based on their homology to the nucleic acids disclosed herein using these nucleic acids or a suitable fragment thereof, as a hybridization probe according to standard hybridization techniques, preferably under highly stringent hybridization conditions. Alternatively, one could apply in silico screening through the available genome databases. “Stringency” of hybridization reactions is readily determinable by one of ordinary skill in the art. For additional details and explanation of stringency of hybridization reactions, see Ausubel et al. (1995, Current Protocols in Molecular Biology, Wiley Interscience Publishers).

The nucleic acid sequence may be isolated by e.g. screening a genomic or cDNA library of the microorganism in question. Once a nucleic acid sequence encoding a polypeptide having an activity according to the invention has been detected with e.g. a probe derived from SEQ ID NO 2 or SEQ ID NO 10, the sequence may be isolated or cloned by utilizing techniques which are known to those of ordinary skill in the art (see Sambrook et al., 1989, Molecular Cloning, A Laboratory Manual, 2d edition, Cold Spring Harbor, N.Y.). The cloning of the nucleic acid sequences of the present invention from such (genomic) DNA can also be effected, e.g. by using methods based on polymerase chain reaction (PCR) or antibody screening of expression libraries to detect cloned DNA fragments with shared structural features (See, e.g., Innis et al., 1990, PCR: A Guide to Methods and Application, Academic Press, New York.).

The sequence information as provided herein should not be so narrowly construed as to require inclusion of erroneously identified bases. The specific sequences disclosed herein can be readily used to isolate the complete gene from ascomycetes, in particular Penicillium chrysogenum, which in turn can easily be subjected to further sequence analyses thereby identifying sequencing errors.

Unless otherwise indicated, all nucleotide sequences determined by sequencing a DNA molecule herein where determined using an automated DNA sequencer and all amino acid sequences of polypeptides encoded by DNA molecules determined herein were predicted by translation of a DNA sequence determined as above. Therefore, as is known in the art for any DNA sequence determined by this approach, any nucleotide sequence determined herein may contain errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion. The person skilled in the art is capable of identifying such erroneously identified bases and knows how to correct for such errors.

In a fourth embodiment the invention provides for alternative HMGR enzymes like the polypeptides of SEQ ID NO 22, SEQ ID NO 26 or SEQ ID NO 30, respectively obtained from the natural statin producers Penicillium citrinum, Monascus pilosus and Aspergillus terreus. The scope of this invention is not limited to these specific amino acid sequences, but includes polypeptide variants with an “improved catalytic function”. Specific examples are polypeptides having specific amino acid mutations making these enzymes more resistant towards statins. Also provided are the DNA sequences encoding these enzymes (SEQ ID NO 19 or 20, SEQ ID NO 23 or 24, SEQ ID NO 27 or 28). The scope of this invention is not limited to these specific nucleotide sequences, but includes “improved genes”. Specific examples are codon pair optimized coding sequences (respectively SEQ ID NO 21, SEQ ID NO 25, and SEQ ID NO 29) and substantial homologous sequences thereof. A polynucleotide with a nucleotide sequence that is substantially homologous to SEQ ID NO 21 is defined as a polynucleotide with a nucleotide sequence with a degree of identity to the specified nucleotide sequence of at least 80%, preferably at least 85%, more preferably at least 90%, still more preferably at least 95%, still more preferably at least 97%, still more preferably at least 98%, most preferably at least 99%. A polynucleotide with a nucleotide sequence that is substantially homologous to SEQ ID NO 25 is defined as a polynucleotide with a nucleotide sequence with a degree of identity to the specified nucleotide sequence of at least 85%, more preferably at least 90%, still more preferably at least 95%, still more preferably at least 97%, still more preferably at least 98%, most preferably at least 99%. A polynucleotide with a nucleotide sequence that is substantially homologous to SEQ ID NO 29 is defined as a polynucleotide with a nucleotide sequence with a degree of identity to the specified nucleotide sequence of at least 80%, preferably at least 85%, more preferably at least 90%, still more preferably at least 95%, still more preferably at least 97%, still more preferably at least 98%, most preferably at least 99%.

In a second aspect, the present invention discloses the use of a polynucleotide of the first aspect in recombinant host strains. More particularly, disclosed is a method for producing compactin, pravastatin, lovastatin and/or simvastatin, comprising the steps of:
(i) transforming a host cell of interest with a polynucleotide comprising the gene of interest encoding HMGR;
(ii) selecting clones of transformed cells;
(iii) optionally, transforming the cells of (ii) with one or more polynucleotides comprising gene(s) encoding key steps in the biosynthesis of compactin, pravastatin, lovastatin and/or simvastatin (i.e. ‘statin biosynthetic genes’);
(iv) cultivating said selected cells, and
(v) isolating compactin, pravastatin, lovastatin and/or simvastatin from said cultivations.

In a preferred embodiment the cell contains all genetic information to produce compactin, pravastatin, lovastatin and/or simvastatin (i.e. ‘statin biosynthetic genes’) from the raw feed stocks supplied during fermentation. Alternatively, precursors may be fed to the cells during step (iv) to produce compactin, pravastatin, lovastatin and/or simvastatin (like activated dimethylbutyric acid and/or monacolin J to produce simvastatin; or compactin to produce pravastatin). The host of step (i) may or may not contain one or more polynucleotides comprising gene(s) encoding key steps in the biosynthesis of compactin, pravastatin, lovastatin and/or simvastatin.

The choice of a host cell in the method of the present invention will to a large extent depend upon the source of the nucleic acid sequence (gene) of interest encoding a polypeptide. Preferably, the host cell is a fungal cell, such as Saccharomyces, Aspergillus or Penicillium species, suitable examples of which are the yeast Saccharomyces cerevisiae or the filamentous fungi Aspergillus niger, Penicillium chrysogenum or Penicillium citrinum. Alternatively, a prokaryotic host cell can be used, examples of which are, but are not limited to, Streptomyces species (i.e. Streptomyces carbophilus, Streptomyces flavidovirens, Streptomyces coelicolor, Streptomyces lividans, Streptomyces exfoliatus) or Amycolatopsis species (i.e. Amycolatopsis orientalis). In a preferred situation, the prokaryotic host cell is a host cell suitable for large scale fermentation, examples of which are, but are not limited to, Streptomyces species (i.e. Streptomyces avermitilis, Streptomyces lividans, Streptomyces clavuligerus) or Bacillus species (i.e. Bacillus subtilus, Bacillus amyloliquefaciens, Bacillus licheniformis) or Corynebacterium species (i.e. Corynebacterium glutamicum) or Escherichia species (i.e. Escherichia coli).

In a preferred embodiment the HMGR encoding genes (SEQ ID NO 1, 2, 3, 9, 10 or 11), all natural HMGR sequences and functional equivalents (SEQ ID NO 19, 20, 21, 23, 24, 25, 27, 28 or 29) can be expressed in a compactin, pravastatin, lovastatin and/or simvastatin producing host cell. Preferably, one should retransform the modified host with one or more genes encoding key steps in the biosynthesis of compactin, pravastatin, lovastatin and/or simvastatin to maximize the productivity in strains with over expressed HMGR. Alternatively, one could start with a non-producing host and first over express HMGR before introducing biosynthetic genes of compactin, pravastatin, lovastatin and/or simvastatin.

In case of a eukaryotic host cell one preferably adapts the expression constructs towards efficient expression in such hosts. Preferably, the host cell is a fungus, more preferably a filamentous fungus, most preferably, the fungal host cell is a cell which produces statins, preferably compactin. Examples of which are, but are not limited to, Aspergillus species (i.e. Aspergillus terreus), or Penicillium species (i.e. Penicillium citrinum or chrysogenum), or Monascus species (i.e. Monascus ruber or paxii).

Nucleic acid constructs, e.g. expression constructs, may contain a selection marker gene and the polynucleotide of the invention (HMGR), each operably linked to one or more control sequences, which direct the expression of the encoded polypeptide in a suitable expression host. The nucleic acid constructs may be on separate fragments or, preferably, on one DNA fragment. Expression will be understood to include any step involved in the production of the polypeptide and may include transcription, post-transcriptional modification, translation, post-translational modification and secretion. The term “nucleic acid construct” is synonymous with the term “expression vector” or “cassette” when the nucleic acid construct contains all the control sequences required for expression of a coding sequence in a particular host organism. The term “control sequences” is defined herein to include all components, which are necessary or advantageous for the expression of a polypeptide. Each control sequence may be native or foreign to the nucleic acid sequence encoding the polypeptide. Such control sequences may include, but are not limited to, a promoter, a leader, optimal translation initiation sequences (as described in Kozak, J. Biol. Chem. 1991, 266:19867-19870), a secretion signal sequence, a pro-peptide sequence, a polyadenylation sequence, a transcription terminator. At a minimum, the control sequences include a promoter, and transcriptional and translational stop signals. The term “operably linked” is defined herein as a configuration in which a control sequence is appropriately placed at a position relative to the coding sequence of the DNA sequence such that the control sequence directs the production of a polypeptide.

The control sequence may include an appropriate promoter sequence containing transcriptional control sequences. The promoter may be any nucleic acid sequence, which shows transcription regulatory activity in the cell including mutant, truncated, and hybrid promoters, and may be obtained from genes encoding extra cellular or intracellular polypeptides. The promoter may be either homologous or heterologous to the cell or to the polypeptide. Preferred promoters for prokaryotic cells are known in the art and can be, for example, strong promoters ensuring high level messenger RNA.

Preferred promoters for filamentous fungal cells are known in the art and can be, for example, the glucose-6-phosphate dehydrogenase gpdA promoters, protease promoters such as pepA, pepB, pepC, the glucoamylase glaA promoters, amylase amyA, amyB promoters, the catalase catR or catA promoters, glucose oxidase goxC promoter, beta-galactosidase lacA promoter, alpha-glucosidase aglA promoter, translation elongation factor tefA promoter, xylanase promoters such as xlnA, xlnB, xlnC, xlnD, cellulase promoters such as eglA, eglB, cbhA, promoters of transcriptional regulators such as areA, creA, xlnR, pacC, prtT, etc or any other, and can be found among others at the NCBI website (http://www.ncbi.nlm.nih.gov/entrez/).

In a preferred embodiment, to obtain over expression, the promoter may be derived from a gene, which is highly expressed (defined herein as the mRNA concentration with at least 0.5% (w/w) of the total cellular mRNA). In another preferred embodiment, the promoter may be derived from a gene, which is medium expressed (defined herein as the mRNA concentration with at least 0.01% until 0.5% (w/w) of the total cellular mRNA). In another preferred embodiment, the promoter may be derived from a gene, which is low expressed (defined herein as the mRNA concentration lower than 0.01% (w/w) of the total cellular mRNA).

In an even more preferred embodiment, Micro Array data is used to select genes, and thus promoters of those genes, that have a certain transcriptional level and regulation. In this way one can adapt the gene expression cassettes optimally to the conditions it should function in.

The control sequence may also include a suitable transcription terminator sequence, a sequence recognized by a filamentous fungal cell to terminate transcription. The terminator sequence is operably linked to the 3′-terminus of the nucleic acid sequence encoding the polypeptide. Any terminator, which is functional in the cell, may be used in the present invention. Preferred terminators for filamentous fungal cells are obtained from the genes encoding Aspergillus oryzae TAKA amylase, Aspergillus niger glucoamylase, Aspergillus nidulans anthranilate synthase, Aspergillus niger alpha-glucosidase, trpC gene and Fusarium oxysporum trypsin-like protease.

The control sequence may also include a suitable leader sequence, a non-translated region of an mRNA, which is important for translation by the filamentous fungal cell. The leader sequence is operably linked to the 5′-terminus of the nucleic acid sequence encoding the polypeptide. Any leader sequence, which is functional in the cell, may be used in the present invention. Preferred leaders for filamentous fungal cells are obtained from the genes encoding Aspergillus oryzae TAKA amylase and Aspergillus nidulans triose phosphate isomerase and Aspergillus niger glaA.

The control sequence may also include a polyadenylation sequence, operably linked to the 3′-terminus of the nucleic acid sequence and which, when transcribed, is recognized by the filamentous fungal cell as a signal to add polyadenosine residues to transcribed mRNA. Any polyadenylation sequence, functional in the cell, may be used in the present invention. Preferred polyadenylation sequences for filamentous fungal cells are obtained from the genes encoding Aspergillus oryzae TAKA amylase, Aspergillus niger glucoamylase, Aspergillus nidulans anthranilate synthase, Fusarium oxysporum trypsin-like protease and Aspergillus niger α-glucosidase.

For secretion of a polypeptide, the control sequence may include a signal peptide-encoding region, coding for an amino acid sequence linked to the amino terminus of the polypeptide, which can direct the encoded polypeptide into the cell's secretory pathway. The 5′-end of the coding sequence may inherently contain a signal peptide-coding region naturally linked in translation reading frame with the segment of the coding region, encoding the secreted polypeptide. Alternatively, the 5′-end of the coding sequence may contain a signal peptide-coding region, foreign to the coding sequence. The foreign signal peptide-coding region may be required where the coding sequence does not normally contain a signal peptide-coding region. Alternatively, the foreign signal peptide-coding region may simply replace the natural signal peptide-coding region in order to obtain enhanced secretion of the polypeptide.

The nucleic acid construct may be an expression vector. The expression vector may be any vector (e.g. a plasmid or virus), which can be conveniently subjected to recombinant DNA procedures and can bring about the expression of the nucleic acid sequence encoding the polypeptide. The choice of the vector will typically depend on the compatibility of the vector with the cell into which the vector is to be introduced. The vectors may be linear or closed circular plasmids.

The vector may be an autonomously replicating vector, i.e. a vector, existing as an extrachromosomal entity, the replication of which is independent of chromosomal replication, e.g. a plasmid, an extrachromosomal element, a minichromosome, or an artificial chromosome. An autonomously maintained cloning vector for a filamentous fungus may comprise the AMA1-sequence (see e.g. Aleksenko and Clutterbuck, Fungal Genet. Biol. 1997, 21: 373-397). Alternatively, the vector may be one which, when introduced into the cell, is integrated into the genome and replicated together with the chromosome(s) into which it has been integrated. The integrative cloning vector may integrate at random or at a predetermined target locus in the chromosomes of the host cell. Preferably, the integrative cloning vector comprises a DNA fragment, which is homologous to a DNA sequence in a predetermined target locus in the genome of host cell for targeting the integration of the cloning vector to this predetermined locus. In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is preferably at least at least 0.1 kb, even preferably at least 0.2 kb, more preferably at least 0.5 kb, even more preferably at least 1 kb, most preferably at least 2 kb. The vector system may be a single vector or plasmid or two or more vectors or plasmids, which together contain the total DNA to be introduced into the genome of the host cell.

The DNA constructs may be used on an episomal vector. Preferably, the constructs are integrated in the genome of the host strain.

Fungal cells are transformed using co-transformation, i.e. along with gene(s) of interest also a selectable marker gene is transformed. This can be either physically linked to the gene of interest (i.e. on a plasmid) or on a separate fragment. Following transfection transformants are screened for the presence of this selection marker gene and subsequently analyzed for the presence of the gene(s) of interest. A selectable marker is a product, which provides resistance against a biocide or virus, resistance to heavy metals, prototrophy to auxotrophs and the like. Useful selectable markers include amdS (acetamidase), argB (ornithinecarbamoyltransferase), bar (phosphinothricinacetyl-transferase), hygB (hygromycin phosphotransferase), niaD (nitrate reductase), pyrG (orotidine-5′-phosphate decarboxylase), sC or sutB (sulfate adenyltransferase), trpC (anthranilate synthase), ble (phleomycin resistance protein), or equivalents thereof.

The obtained host cell may be used for producing compactin, pravastatin, lovastatin and/or simvastatin.

In a third aspect, the present invention provides a host cell used in the second aspect comprising the polynucleotide of the first aspect of the invention. The host cell of the second aspect may be further improved by various approaches.

In one embodiment, the application of the polypeptides of the present invention can be improved by deleting one or more of the endogenous genes from the genome of the host strain encoding enzymes limiting compactin, pravastatin, lovastatin and/or simvastatin yields. Examples of such enzymes are, but are not limited to, enzymes that hydrolyze the side chains of compactin, pravastatin, lovastatin and/or simvastatin as for instance described in co-pending application EP07123446.2.

In another embodiment, the compactin, pravastatin, lovastatin and/or simvastatin productivity of the recombinant host cell may be improved via classical mutagenesis.

In yet another embodiment, resistance versus and/or productivity of compactin, pravastatin, lovastatin and/or simvastatin may be further improved by re-transforming with genes not encoding HMGR. Examples are efflux proteins or transporter proteins.

In a fourth aspect of the present invention, the compactin, pravastatin, lovastatin and/or simvastatin produced according to the method of the second and third aspect is comprised within a pharmaceutical composition.

### LEGENDS TO THE FIGURES

FIG. 1 shows a representation of the steps involved in deleting a Penicillium chrysogenum gene, for example SEQ ID NO 1. Legend: solid arrow, promoter; open box, gene-of-interest; open arrow, terminator; hatched box, trpC terminator; dashed box, ccdA gene; solid box, lox site; crosses, recombination event; downwards arrows, subsequent steps in the procedure; REKR and KRAM, overlapping non-functional amdS selection marker fragments; REKRAM, functional amdS selection marker gene. Numbers indicate the SEQ ID NO's of the oligonucleotides.

### SEQUENCE LISTING FREE TEXT

### SEQ ID NO 3: Synthetic DNA

### SEQ ID NO 5: Oligonucleotide

### SEQ ID NO 6: Oligonucleotide

### SEQ ID NO 7: Oligonucleotide

### SEQ ID NO 8: Oligonucleotide

### SEQ ID NO 11: Synthetic DNA

### SEQ ID NO 13: Oligonucleotide

### SEQ ID NO 14: Oligonucleotide

### SEQ ID NO 15: Oligonucleotide

### SEQ ID NO 16: Oligonucleotide

### SEQ ID NO 17: Oligonucleotide

### SEQ ID NO 18: Oligonucleotide

### SEQ ID NO 21: Synthetic DNA

### SEQ ID NO 25: Synthetic DNA

### SEQ ID NO 29: Synthetic DNA

SEQ ID NO 33: PCR amplified from plasmid DNA
SEQ ID NO 34: PCR amplified from plasmid DNA
SEQ ID NO 37: PCR amplified from plasmid DNA
SEQ ID NO 38: PCR amplified from plasmid DNA
SEQ ID NO 39: Plasmid DNA sequence
SEQ ID NO 40: Plasmid DNA sequence

### EXAMPLES

### General Materials and Methods

Standard DNA procedures were carried out as described elsewhere (Sambrook, J. et al., 1989, Molecular cloning: a laboratory manual, 2^{nd} Ed., Cold Spring Harbor Laboratory Press, Cold Spring Harbor, N.Y.) unless otherwise stated. DNA was amplified using the proofreading enzyme Physion polymerase (Finnzymes). Restriction enzymes were from Invitrogen or New England Biolabs.

Fungal growth was performed in a mineral medium, containing (g/L): glucose (5); lactose (35); urea (4.5); (NH₄)₂SO₄ (1.1); Na₂SO₄ (2.9); KH₂PO₄ (5.2); K₂HPO₄ (4.8) and 10 mL/L of a trace element solution containing (in g/l): citric acid (150); FeSO₄.7H₂O (15); MgSO₄.7H₂O (150); H₃BO₃ (0.0075); CuSO₄.5H₂O (0.24); CoSO₄.7H₂O (0.375); ZnSO₄.7H₂O (5); MnSO₄.H₂O (2.28); CaCl₂.2H₂O (0.99); pH before sterilization 6.5. Oxoid agar was added at 15 g/l to solidify the medium.

Compactin was maintained as a stock solution at 20 g/L in ethanol at −20 C.

Although the Examples given below are illustrative for Penicillium chrysogenum, they are not meant to exclude other organism. In particular the skilled person will be able to repeat the invention for microorganisms such as Aspergillus terreus or Penicillium citrinum.

### Example 1

### Deletion of Penicillium chrysogenum Gene Pc18g05230 (SEQ ID NO 1) Encoding a HMGR Enzyme

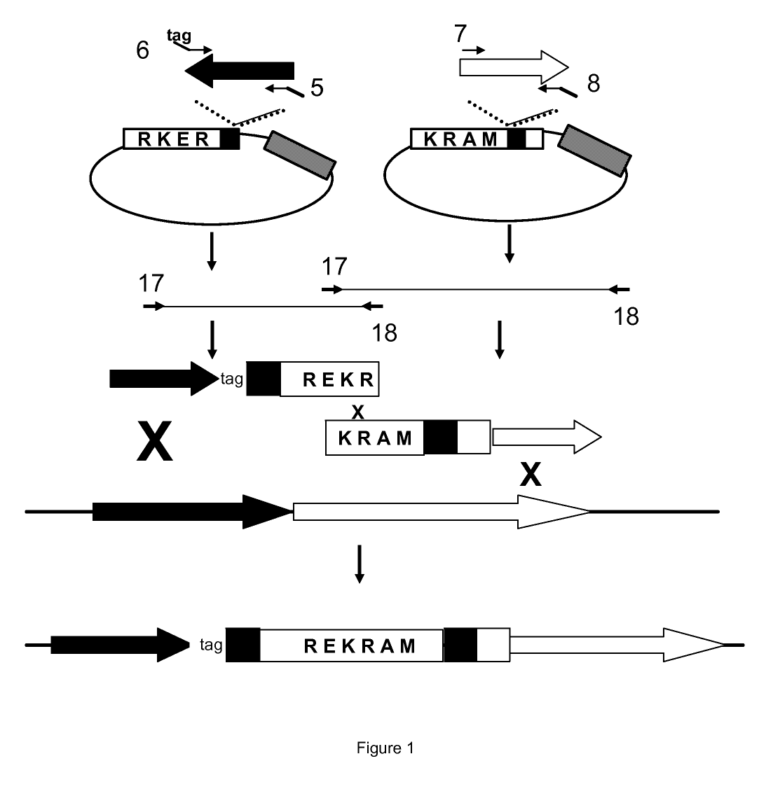

The gene Pc18g05230 was identified as a HMGR encoding gene. In order to prevent the transcription of this gene a selection marker gene was inserted between the promoter and the open reading frame (ORF). To this end the promoter and the ORF were PCR amplified using the oligonucleotides SEQ ID NO 5 plus 6 and SEQ ID NO 7 plus 8, respectively (see FIG. 1). Phusion Hot-Start Polymerase (Finnzymes) was used to amplify the fragments. The fragments obtained are 1539 and 2514 base pairs (bp) in length (SEQ ID NO 31 and SEQ ID NO 32) and contain a 14 bp tail suitable for the so-called STABY cloning method (Eurogentec). From the standard STABY vector, pSTC1.3, two derivatives were obtained. One, pSTamdSL (SEQ ID NO 39), was used for cloning the PCR amplified promoter (SEQ ID NO 31). The other, pSTamdSR (SEQ ID NO 40), was used for cloning the PCR amplified ORF (SEQ ID NO 32). pSTamdSL was constructed by insertion of an inactive part of the amdS selection marker gene (see for example the PgpdA-amdS cassette of pHELY-A1 in WO 2004/106347) by PCR amplification of the last ⅔ of the gene (amdS) and cloning it in the HindIII-BamHI sites of pSTC1.3. pSTamdSR was constructed by insertion of another inactive part of the amdS selection marker gene (see for example the PgpdA-amdS cassette of pHELY-A1 in WO 2004/106347) by PCR amplification of the PgpdA promoter and the first ⅔ of the gene wherein the EcoRV sites where removed and cloning it in the HindIII-PmeI sites of pSTC1.3. Also, a strong terminator was inserted in front of the PgpdA-amdS; the trpC terminator was PCR amplified and introduced via the SbfI-NotI sites of the PgpdA-amdS fragment. Both vectors do contain an overlapping but non-functional fragment of the fungal selection marker gene amdS, encoding acetamidase and allowing recipient cells that recombine the two fragments into a functional selection marker to grow on agar media with acetamide as the sole nitrogen source (EP 635574; WO 97/06261; Tilburn et al., 1983, Gene 26: 205-221). The promoter and ORF PCR fragments (SEQ ID NO 31 and SEQ ID NO 32) were ligated into the vectors overnight using T4 ligase (Invitrogen) at 16° C., according to the STABY-protocol (Eurogentec) and transformed to chemically competent CYS21 cells (Eurogentec). Ampicillin resistant clones were isolated and used to PCR amplify the cloned fragments fused to the non-functional amdS fragments (see FIG. 1). This was done using the oligonucleotides SEQ ID NO 17 and SEQ ID NO 18. The thus obtained PCR fragments (SEQ ID NO 33 and 34) were combined and used to transform a derivative of Penicillium chrysogenum strain DS17690 (S917) deposited at the Centraalbureau voor Schimmelcultures, Utrecht, The Netherlands on Apr. 15, 2008 with deposition number CBS122850 with the hdfA gene deleted (according to the method disclosed in WO 2005/095624).

In this strain the non-homologous end-joining pathway is disturbed and therefore the random integration of DNA is drastically reduced. And as the combined PCR fragments themselves should recombine also to form a functional amdS selection marker gene (i.e. the so-called bipartite or split-marker method), correct targeted integrants should undergo a triple homologous recombination event (see FIG. 1). More than five transformants were obtained on acetamide containing agar (WO 2008/000715) and one was subsequently transferred to a second acetamide selection plate.

### Example 2

### Deletion of Penicillium chrysogenum Gene Pc16g05060 (SEQ ID NO 9) Encoding a HMGR Enzyme

The gene Pc16g05060 was identified as a HMGR encoding gene. In order to prevent the transcription of this gene a selection marker gene was inserted between the promoter and the open reading frame (ORF). To this end the promoter and the ORF were PCR amplified using the oligonucleotides SEQ ID NO 13 plus 14 and SEQ ID NO 15 plus 16, respectively (see FIG. 1). Phusion Hot-Start Polymerase (Finnzymes) was used to amplify the fragments. The fragments obtained are 1539 and 1514 base pairs (bp) in length (SEQ ID NO 35 and SEQ ID NO 36) and contain a 14 bp tail suitable for the so-called STABY cloning method (Eurogentec).

From the standard STABY vector, pSTC1.3, two derivatives were obtained. One, pSTamdSL (SEQ ID NO 39), was used for cloning the PCR amplified promoter (SEQ ID NO 35). The other, pSTamdSR (SEQ ID NO 40), was used for cloning the PCR amplified ORF (SEQ ID NO 36). pSTamdSL was constructed by insertion of an inactive part of the amdS selection marker gene (see for example the PgpdA-amdS cassette of pHELY-A1 in WO04106347) by PCR amplification of the last ⅔ of the gene (amdS) and cloning it in the HindIII-BamHI sites of pSTC1.3. pSTamdSR was constructed by insertion of another inactive part of the amdS selection marker gene (see for example the PgpdA-amdS cassette of pHELY-A1 in WO 04106347) by PCR amplification of the PgpdA promoter and the first ⅔ of the gene wherein the EcoRV sites where removed and cloning it in the HindIII-PmeI sites of pSTC1.3. Also, a strong terminator was inserted in front of the PgpdA-amdS; the trpC terminator was PCR amplified and introduced via the SbfI-NotI sites of the PgpdA-amdS fragment. Both vectors do contain an overlapping but non-functional fragment of the fungal selection marker gene amdS, encoding acetamidase and allowing recipient cells that recombine the two fragments into a functional selection marker to grow on agar media with acetamide as the sole nitrogen source (EP 635,574; WO97/06261; Tilburn et al., 1983, Gene 26: 205-221). The promoter and ORF PCR fragments (SEQ ID NO 35 and SEQ ID NO 36) were ligated into the vectors overnight using T4 ligase (Invitrogen) at 16° C., according to the STABY-protocol (Eurogentec) and transformed to chemically competent CYS21 cells (Eurogentec). Ampicillin resistant clones were isolated and used to PCR amplify the cloned fragments fused to the non-functional amdS fragments (see FIG. 1). This was done using the oligonucleotides SEQ ID NO 17 and SEQ ID NO 18. The thus obtained PCR fragments (SEQ ID NO 37 and 38) were combined and used to transform a derivative of Penicillium chrysogenum strain DS17690 (S917) deposited at the Centraalbureau voor Schimmelcultures, Utrecht, The Netherlands on Apr. 15, 2008 with deposition number CBS122850 with the hdfA gene deleted (according to the method disclosed in WO 2005/095624). In this strain the non-homologous end-joining pathway is disturbed and therefore the random integration of DNA is drastically reduced. And as the combined PCR fragments themselves should recombine also to form a functional amdS selection marker gene (i.e. the so-called bipartite or split-marker method), correct targeted integrants should undergo a triple homologous recombination event (see FIG. 1).

More than 5 transformants were obtained on acetamide containing agar (WO 2008/000715) and one was subsequently transferred to a second acetamide selection plate.

### Example 3

### Deletion of the Penicillium chrysogenum HMGR Encoding Genes Leads to Increased Compactin Sensitivity

Spores of the Penicillium chrysogenum mutants for both HMGR encoding genes, Pc18g05230 (SEQ ID NO 1) and Pc16g05060 (SEQ ID NO 9), were inoculated in liquid media with different amounts of compactin. After 2 days of growth at 25 C both the mutants clearly showed increased compactin sensitivity (see Table 1), illustrating the role of HMGR enzyme levels.

TABLE 1
Compactin sensitivity of different Penicillium chrysogenum strains

| | Compactin (mg/l) | | | | | | |
|---|---|---|---|---|---|---|---|
| Strain | 0 | 0.25 | 1.0 | 2.5 | 10 | 60 | 200 |
| ΔhdfA | +++ | +++ | +++ | +++ | +++ | +++ | −−− |
| ΔhdfAΔ Pc18g05230 | +++ | +++ | +++ | +++ | +++ | −−− | −−− |
| ΔhdfAΔ Pc16g05060 | +++ | +++ | +++ | +++ | +++ | −−− | −−− |

### Example 4

### Over Expression of the Penicillium chrysogenum HMGR Encoding Genes

In order to overexpress the HMGR activity, a strong promoter should be inserted between the original promoter and the open reading frame (ORF) of Pc18g05230 (SEQ ID NO 1) and Pc16g05060 (SEQ ID NO 9). Basically the same PCR-amplified fragments (i.e. promoter and ORF) of examples 1 and 2 will be used. However, to drive over expression the ORF should be cloned in a variant vector of pSTamdSR, which contains a strong promoter in front of the trpC terminator. Further steps (i.e. cloning, 2nd PCR, transformation, selection of transformants) are as in examples 1 and 2. The strains obtained will be tested for compactin resistance.

### Example 5

### Over Expression of the HMGR Encoding Genes in Compactin/Pravastatin Producing Penicillium chrysogenum

In order to overexpress the HMGR activity in a statin producing host, the bipartite fragments of example 4 are transfected to a statin producing host. Further steps (i.e. selection of transformants) are as in examples 1 and 2. The strains obtained will be tested for compactin/pravastatin productivity.

1. Polypeptide with HMG-CoA reductase activity chosen from the group consisting of SEQ ID 4, SEQ ID 12, a polypeptide with an amino acid sequence with a degree of identity to SEQ ID 4 of at least 80% and a polypeptide with an amino acid sequence with a degree of identity to SEQ ID 12 of at least 60%.

2. Polynucleotide chosen from the group consisting of SEQ ID 1, SEQ ID 2, SEQ ID 3, SEQ ID 9, SEQ ID 10, SEQ ID 11, SEQ ID 21, SEQ ID 25, SEQ ID 29, a polynucleotide with a sequence with a degree of identity to SEQ ID 1 of at least 80%, a polynucleotide with a sequence with a degree of identity to SEQ ID 2 of at least 80%, a polynucleotide with a sequence with a degree of identity to SEQ ID 3 of at least 85%, a polynucleotide with a sequence with a degree of identity to SEQ ID 9 of at least 60%, a polynucleotide with a sequence with a degree of identity to SEQ ID 10 of at least 60%, a polynucleotide with a is sequence with a degree of identity to SEQ ID 11 of at least 60%, a polynucleotide with a sequence with a degree of identity to SEQ ID 21 of at least 80%, a polynucleotide with a sequence with a degree of identity to SEQ ID 25 of at least 85% and a polynucleotide with a sequence with a degree of identity to SEQ ID 29 of at least 80%.

3. Method for the production of a statin comprising over expression of a polypeptide chosen from the group consisting of SEQ ID 4, SEQ ID 12, SEQ ID 26, SEQ ID 30, a polypeptide with an amino acid sequence with a degree of identity to SEQ ID 4 of at least 80%, a polypeptide with an amino acid sequence with a degree of identity to SEQ ID 12 of at least 60%, a polypeptide with an amino acid sequence with a degree of identity to SEQ ID 26 of at least 70% and a polypeptide with an amino acid sequence with a degree of identity to SEQ ID 30 of at least 70%.

4. Method according to claim 3 comprising the steps of:
(i) transforming a host cell of interest with a polynucleotide comprising the gene of interest encoding HMGR;
(ii) selecting clones of transformed cells;
(iii) cultivating said selected cells, and
(iv) isolating compactin, pravastatin, lovastatin and/or simvastatin from said cultivations.

5. Method according to claim 3 wherein said host cell is transformed with one or more statin biosynthetic genes.

6. Host cell comprising the polynucleotide of claim 2.

7. Host cell according to claim 6, which is a fungus from the genera Penicillium, Aspergillus, Monascus, Mucor or Saccharomyces.

8. Host cell according to claim 6 wherein said host cell is Penicillium citrinum, Penicillium chrysogenum, Aspergillus niger, Aspergillus terreus, Aspergillus nidulans, Monascus tuber, Monascus paxi, Mucor hiemalis or Saccharomyces cerevisiae.

9. Host cell which is Penicillium chrysogenum comprising the polynucleotide of claim 2 or a polynucleotide chosen from the group consisting of SEQ ID 19, SEQ ID 20, SEQ ID 23, SEQ ID 24, SEQ ID 27 and SEQ ID 28.

10. Use of the pravastatin, lovastatin and/or simvastatin obtained in claim 3 in the production of a medicament.
